(19) United States
(12) Patent Application Publication
(10) Pub. No.: US 2014/0114194 A1
(43) Pub. Date: Apr. 24, 2014

(54) ULTRASOUND DIAGNOSIS APPARATUS AND ULTRASOUND PROBE CONTROLLING METHOD

(71) Applicants: Toshiba Medical Systems Corporation, Otawara-shi (JP); Kabushiki Kaisha Toshiba, Minato-ku (JP)

(72) Inventors: Yuko KANAYAMA, Nasushiobara-shi (JP); Naohisa KAMIYAMA, Utsunomiya-shi (JP)

(73) Assignees: Toshiba Medical Systems Corporation, Otawara-shi (JP); Kabushiki Kaisha Toshiba, Minato-ku (JP)

(21) Appl. No.: 14/144,763

(22) Filed: Dec. 31, 2013

Related U.S. Application Data
(63) Continuation of application No. PCT/JP2012/067097, filed on Jul. 4, 2012.

(30) Foreign Application Priority Data
Jul. 4, 2011 (JP) 2011-148299
Jul. 3, 2012 (JP) 2012-149489

(51) Int. Cl. A61B 8/00; A61B 8/14; A61B 8/08

(52) U.S. Cl. CPC A61B 8/4254; A61B 8/4444; A61B 8/429; A61B 8/5207; A61B 8/14; A61B 8/461
USPC 600/459

(57) ABSTRACT

An ultrasound diagnosis apparatus according to an embodiment includes a detecting unit and a deflecting unit. The detecting unit detects at least one selected from between a force applied to an ultrasound probe and a movement of the ultrasound probe. The deflecting unit tilts a transmission direction of an ultrasound wave transmitted from the ultrasound probe, based on at least one selected from between the force and the movement.

### CROSS-REFERENCE TO RELATED APPLICATIONS

This application is a continuation of International Application No. PCT/JP2012/067097, filed on Jul. 4, 2012 which claims the benefit of priority of the prior Japanese Patent Application No. 2011-148299, filed on Jul. 4, 2011, and Japanese Patent Application No. 2012-149489, filed on Jul. 3, 2012, the entire contents of which are incorporated herein by reference.

### FIELD

Embodiments described herein relate generally to an ultrasound diagnosis apparatus and an ultrasound probe controlling method.

### BACKGROUND

Conventionally, ultrasound diagnosis apparatuses have been used in present-day medical care for performing medical examinations and making diagnoses on various tissues in patients' bodies such as the heart, liver, kidneys, mammary glands, and the like, as ultrasound diagnosis apparatuses have advantages over other medical image diagnosis apparatuses such as X-ray diagnosis apparatuses and X-ray computed tomography apparatuses, due to simpler operability and due to being non-invasive while having no possibility of causing radiation exposure. Such an ultrasound diagnosis apparatus is configured to generate an ultrasound image, which is an image of a tissue structure on the inside of an examined subject (hereinafter, “patient”), by transmitting ultrasound waves from an ultrasound probe and receiving a reflected-wave signal reflected by internal tissues of the patient. In that situation, the ultrasound diagnosis apparatus generates the ultrasound image in such a manner that the closer to perpendicular the angle of the irradiation of the ultrasound waves is, the more clearly the irradiated tissue is rendered.

### BRIEF DESCRIPTION OF THE DRAWINGS

FIG. 1 is a block diagram of an exemplary configuration of an ultrasound diagnosis apparatus according to a first embodiment;

FIG. 2 is a drawing of examples of ultrasound images generated by an ultrasound diagnosis apparatus;

FIG. 3 is a diagram of an exemplary configuration of a controlling unit and the like according to the first embodiment;

FIG. 4 is a drawing of examples of processes performed by the controlling unit according to the first embodiment;

FIG. 5 is a drawing of an example of an ultrasound image generated by the ultrasound diagnosis apparatus according to the first embodiment;

FIG. 6 is a flowchart of a processing procedure performed by the ultrasound diagnosis apparatus according to the first embodiment;

FIG. 7 is a diagram of an exemplary configuration of a controlling unit and the like according to a second embodiment;

FIG. 8 is a drawing of examples of processes performed by the controlling unit according to the second embodiment; and

FIG. 9 is a flowchart of a processing procedure performed by an ultrasound diagnosis apparatus according to the second embodiment.

### DETAILED DESCRIPTION

An ultrasound diagnosis apparatus according to an embodiment includes a detecting unit and a deflecting unit. The detecting unit detects at least one selected from between a force applied to an ultrasound probe and a movement of the ultrasound probe. The deflecting unit tilts a transmission direction of an ultrasound wave transmitted from the ultrasound probe, based on at least one selected from between the force and the movement.

### First Embodiment

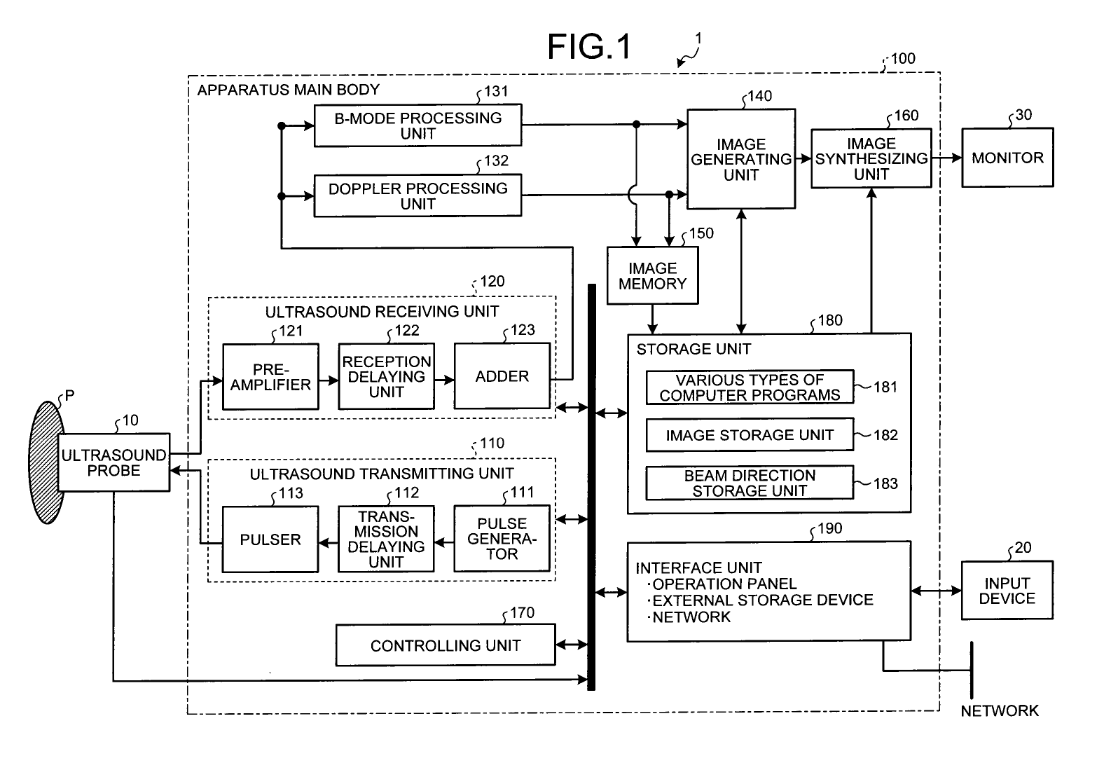

First, a configuration of an ultrasound diagnosis apparatus according to a first embodiment will be explained. FIG. 1 is a block diagram of an exemplary configuration of the ultrasound diagnosis apparatus according to the first embodiment. As shown in FIG. 1, an ultrasound diagnosis apparatus 1 according to the first embodiment includes an ultrasound probe 10, an input device 20, a monitor 30, and an apparatus main body 100.

The ultrasound probe 10 includes a plurality of piezoelectric vibrators, which generate ultrasound waves based on a drive signal supplied from an ultrasound transmitting unit 110 included in the apparatus main body 100 (explained later). Further, the ultrasound probe 10 receives a reflected-wave signal from a patient P and converts the received reflected-wave signal into an electric signal. Further, the ultrasound probe 10 includes matching layers included in the piezoelectric vibrators, as well as a backing member that prevents ultrasound waves from propagating rearward from the piezoelectric vibrators. The ultrasound probe 10 is detachably connected to the apparatus main body 100.

When an ultrasound wave is transmitted from the ultrasound probe 10 to the patient P, the transmitted ultrasound wave is repeatedly reflected on a surface of discontinuity of acoustic impedances at a tissue in the body of the patient P and is received as the reflected-wave signal by the plurality of piezoelectric vibrators included in the ultrasound probe 10. The amplitude of the received reflected-wave signal is dependent on the difference between the acoustic impedances on the surface of discontinuity on which the ultrasound wave is reflected. When the transmitted ultrasound pulse is reflected on the surface of a flowing bloodstream or a cardiac wall, the reflected-wave signal is, due to the Doppler effect, subject to a frequency shift, depending on a velocity component of the moving members with respect to the ultrasound wave transmission direction.

The input device 20 is connected to the apparatus main body 100 and includes a mouse, a keyboard, a button, a panel switch, a touch command screen, a foot switch, a trackball, and the like. The input device 20 receives various types of setting requests from an operating person (hereinafter, “operator”) of the ultrasound diagnosis apparatus 1 and transfers the received various types of setting requests to the apparatus main body 100.

The monitor 30 displays a Graphical User Interface (GUI) used by the operator of the ultrasound diagnosis apparatus 1 to input the various types of setting requests through the input device 20 and displays an ultrasound image and the like generated by the apparatus main body 100. More specifically, the monitor 30 displays morphological information and bloodstream information within the patient's body as the image, based on a video signal that is input thereto from an image synthesizing unit 160 (explained later).

The apparatus main body 100 generates the ultrasound image based on the reflected-wave signal received by the ultrasound probe 10. As shown in FIG. 1, the apparatus main body 100 includes the ultrasound transmitting unit 110, an ultrasound receiving unit 120, a B-mode processing unit 131, a Doppler processing unit 132, an image generating unit 140, an image memory 150, the image synthesizing unit 160, a controlling unit 170, a storage unit 180, and an interface unit 190.

The ultrasound transmitting unit 110 includes a pulse generator 111, a transmission delaying unit 112, and a pulser 113 and supplies the drive signal to the ultrasound probe 10. The pulse generator 111 repeatedly generates a rate pulse for forming a transmission ultrasound wave at a predetermined rate frequency. Further, the transmission delaying unit 112 applies a delay period that is required to converge the ultrasound wave generated by the ultrasound probe 10 into the form of a beam and to determine transmission directionality and that corresponds to each of the piezoelectric vibrators, to each of the rate pulses generated by the pulse generator 111. Further, the pulser 113 applies a drive signal (a drive pulse) to the ultrasound probe 10 with timing based on the rate pulses. In other words, the transmission delaying unit 112 arbitrarily adjusts the transmission directions of the ultrasound waves transmitted from the piezoelectric vibrator surfaces, by varying the delay periods applied to the rate pulses. The transmission directions or the delay periods that determine the transmission directions are stored in the storage unit 180. The transmission delaying unit 112 may apply the delay periods by referring to the storage unit 180.

The ultrasound receiving unit 120 includes a preamplifier 121, an Analog/Digital (A/D) converter (not shown), a reception delaying unit 122, and an adder 123 and generates reflected-wave data by performing various types of processes on the reflected-wave signal received by the ultrasound probe 10. The preamplifier 121 amplifies the reflected-wave signal for each of channels. The A/D converter (not shown) applies an A/D conversion to the amplified reflected-wave signal. The reception delaying unit 122 applies a delay period required to determine reception directionality. The adder 123 generates the reflected-wave data by performing an adding process on the reflected-wave signal processed by the reception delaying unit 122. As a result of the adding process performed by the adder 123, reflected components from the direction corresponding to the reception directionality of the reflected-wave signal are emphasized, so that comprehensive beams for the ultrasound transmission and reception are formed based on the reception directionality and the transmission directionality. Similarly to the description of the transmission, the reception directions or the delay periods that determine the reception directions are stored in the storage unit 180, so that the reception delaying unit 122 applies the delay periods by referring to the storage unit 180.

The B-mode processing unit 131 receives the reflected-wave data from the ultrasound receiving unit 120 and generates data (B-mode data) in which the strength of each signal is expressed by a degree of brightness, by performing a logarithmic amplification, an envelope detection process, and the like on the received reflected-wave data.

The Doppler processing unit 132 extracts bloodstreams, tissues, and contrast echo components under the influence of the Doppler effect by performing a frequency analysis so as to obtain velocity information from the reflected-wave data received from the ultrasound receiving unit 120, and further generates data (Doppler data) obtained by extracting bloodstream information such as an average velocity, the dispersion, the power, and the like for a plurality of points.

The image generating unit 140 generates ultrasound images from the B-mode data generated by the B-mode processing unit 131 and the bloodstream information generated by the Doppler processing unit 132 and stores the generated ultrasound images into the image memory 150 or the storage unit 180 (explained later).

More specifically, from the B-mode data, the image generating unit 140 generates a B-mode image in which the strength of the reflected-wave data is expressed by a degree of brightness. Also, from the bloodstream information, the image generating unit 140 generates a color Doppler image in which the average velocity and the dispersion of the bloodstream as well as a blood flow amount, and a combination thereof are displayed in a recognizable manner by using colors.

Further, the image generating unit 140 converts (by performing a scan convert process) a scanning line signal sequence from an ultrasound scan into a scanning line signal sequence in a video format used by, for example, television and generates ultrasound images (e.g., a B-mode image and a color Doppler image) serving as display images.

The image memory 150 is a memory for storing therein the ultrasound images generated by the image generating unit 140 and images generated by performing image processing on ultrasound images. For example, after making a diagnosis, an operator of the apparatus is able to fetch any of the images recorded during a medical examination, from the image memory 150. It is possible to play back an image in the manner of a still image or a plurality of images in the manner of a moving picture. Further, the image memory 150 stores therein, as necessary, image brightness signals that have passed through the ultrasound receiving unit 120, other raw data, images obtained via a network, and the like.

The image synthesizing unit 160 generates a synthesized image obtained by synthesizing the ultrasound image generated by the image generating unit 140 with text information of various parameters, a scale graduation, a body mark, and the like. The synthesized image generated by the image synthesizing unit 160 is displayed on the monitor 30.

The controlling unit 170 is a controlling processor (a Central Processing Unit (CPU)) that realizes functions of an information processing apparatus (a computer) and is configured to control the entire processes performed by the ultrasound diagnosis apparatus 1. More specifically, based on various types of instructions and setting requests input by the operator via the input device 20 and various types of computer programs and various types of setting information read from the storage unit 180, the controlling unit 170 controls processes performed by the ultrasound transmitting unit 110, the ultrasound receiving unit 120, the B-mode processing unit 131, the Doppler processing unit 132, the image generating unit 140, and the image synthesizing unit 160. The controlling unit 170 also exercises control so that the monitor 30 displays the ultrasound image and the like stored in the image memory 150.

The storage unit 180 stores therein various types of computer programs 181 to realize ultrasound transmissions and receptions, image processing, and display processing, an image storage unit 182 storing therein the ultrasound images generated by the image generating unit 140, as well as various types of data such as diagnosis information (e.g., patients' IDs, medical doctors' observations), diagnosis protocols, and various types of setting information. The various types of computer programs 181 may include a computer program describing a procedure for performing the same processes as the processes performed by the controlling unit 170. Furthermore, the various types of data stored in the storage unit 180 can be transferred to any external peripheral device via the interface unit 190.

Further, the storage unit 180 includes a beam direction storage unit 183 for storing therein, for example, information related to directions of the ultrasound waves transmitted by the ultrasound probe 10. Because the beam direction storage unit 183 is used by the controlling unit 170, the details thereof will be explained later.

The interface unit 190 is an interface related to the input device 20, an operation panel, another external storage device (not shown), and a network. Data such as the ultrasound images obtained by the ultrasound diagnosis apparatus 1 can be transferred to another apparatus via the network by the interface unit 190.

The ultrasound transmitting unit 110, the ultrasound receiving unit 120, and the like that are built in the apparatus main body 100 may be configured by using hardware such as an integrated circuit or may be realized by using a computer program that is in the form of a module in the manner of software.

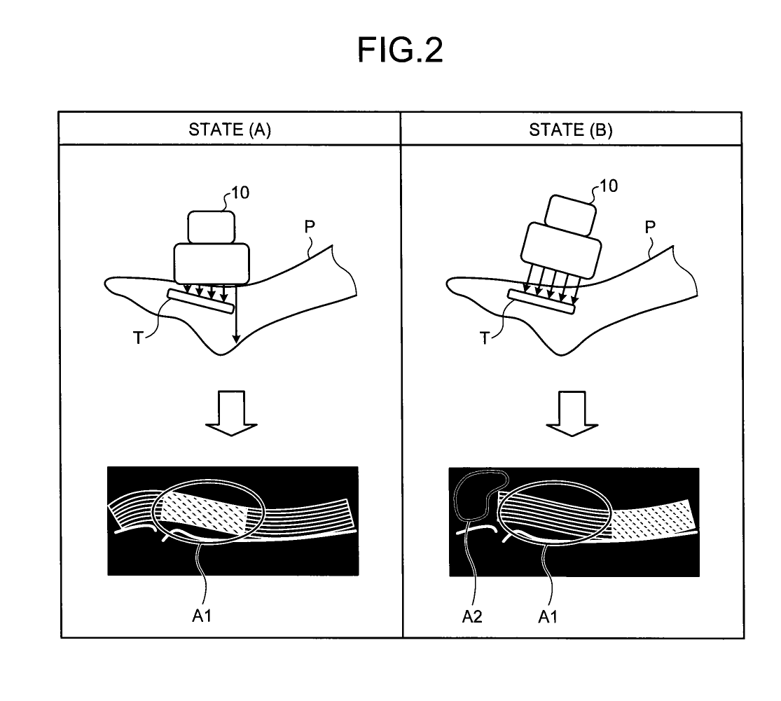

An overall configuration of the ultrasound diagnosis apparatus 1 according to the first embodiment has thus been explained. In this situation, depending on an image taking target of the patient P, a commonly-used ultrasound diagnosis apparatus is not necessarily always able to generate an ultrasound image rendering a tissue which an operator (e.g., a medical doctor) desires to view and other tissues positioned near such a tissue. This aspect will be explained more specifically, with reference to FIG. 2. FIG. 2 is a drawing of examples of ultrasound images generated by an ultrasound diagnosis apparatus. With reference to FIG. 2, examples in which the operator presses the ultrasound probe 10 against an ankle of the patient P will be explained. In other words, in the examples shown in FIG. 2, the ultrasound diagnosis apparatus generates an ultrasound image rendering tissues in the ankle of the patient P. The upper parts of State (A) and State (B) in FIG. 2 illustrate the state in which the ultrasound probe 10 is pressed against the patient P, whereas the lower parts of State (A) and State (B) in FIG. 2 illustrate examples of ultrasound images generated by the ultrasound diagnosis apparatus while the ultrasound probe 10 is in the states shown in the upper parts, respectively.

In this situation, let us assume that the tissue which the operator desires to view is a “target tissue T” shown in FIG. 2. In that situation, in the example shown in the upper part of State (A) in FIG. 2, the target tissue T is not irradiated perpendicularly by the ultrasound waves transmitted by the ultrasound probe 10. For this reason, as shown in an area A1 in the lower part of State (A) in FIG. 2, the ultrasound image generated by the ultrasound diagnosis apparatus may not render the target tissue T clearly. The reason can be explained as follows: In an ultrasound image generated by an ultrasound diagnosis apparatus, the closer to perpendicular the angle of the irradiation of ultrasound waves is, the more clearly the irradiated tissue is rendered.

In that situation, as shown in the example of State (B) in FIG. 2, the operator may perform an operation to tilt the ultrasound probe 10 so that the target tissue T is perpendicularly irradiated by the ultrasound waves, so as to be able to view the target tissue T. As a result of this operation, the target tissue T is substantially perpendicularly irradiated by the ultrasound waves transmitted by the ultrasound probe 10. As a result, as illustrated in the area A1 in the lower part of State (B) in FIG. 2, the ultrasound image generated by the ultrasound diagnosis apparatus renders the target tissue T clearly.

However, when the soft tissue is thin at the site against which the ultrasound probe 10 is pressed, if the ultrasound probe 10 is tilted, the surface of the piezoelectric vibrators (hereinafter, “piezoelectric vibrator surface”) of the ultrasound probe 10 and the surface of the patient's body (hereinafter, “body surface”) may be positioned away from each other, and a gap may be formed between the piezoelectric vibrator surface and the body surface, as shown in the upper part of State (B) in FIG. 2. In that situation, as illustrated in an area A2 in the lower part of State (B) in FIG. 2, the tissue on the inside of the patient P may not be rendered properly in the part corresponding to the gap formed between the piezoelectric vibrator surface and the body surface. In other words, in the example of State (B) in FIG. 2, the tissues positioned near the target tissue T are not properly rendered in the ultrasound image generated by the ultrasound diagnosis apparatus.

In recent years, in orthopedics and the like, a foot, a hand, a joint in a shoulder or a knee, or the like is viewed by using an ultrasound diagnosis apparatus so that a diagnosis can be made regarding the surface of a bone, a muscle rupture, a subcutaneous tumor, a movement of a tendon, or the like. However, because the soft tissue over the bone is thin in a finger and the like, it is difficult for the operator to tilt the ultrasound probe 10 while keeping the ultrasound probe 10 in contact with the body surface. As a result, as illustrated in the example in FIG. 2 described above, the ultrasound diagnosis apparatus may not be able to generate an ultrasound image properly rendering the target tissue T and the tissues positioned near the target tissue T.

Incidentally, another method that makes it possible to tilt the ultrasound probe 10 is also known. According to this method, ultrasound jelly is applied thickly to the body surface at a viewed site. However, this method is not economical due to the use of the ultrasound jelly, and also, the process after the medical examination requires some time and labor, which puts a large burden on the patient P as well. Alternatively, it is also possible to change the directions of the ultrasound waves transmitted from the ultrasound probe 10 by operating the input device 20 (e.g., a knob) included in the ultrasound diagnosis apparatus. This method, however, puts a burden on the operator during a medical examination process to view a large number of sites one after another, because the operator needs to operate the input device 20 many times. In addition, in a situation where the operator is performing a puncture treatment while viewing internal tissues with the ultrasound diagnosis apparatus, the operator is not able to operate the input device 20, because both hands are being used.

To cope with these situations, the ultrasound diagnosis apparatus 1 according to the first embodiment makes it possible to change the transmission directions of the ultrasound waves with an intuitive operation performed by the operator, under the control of the controlling unit 170. More specifically, the controlling unit 170 included in the ultrasound diagnosis apparatus 1 according to the first embodiment is configured to detect a force applied to the ultrasound probe 10 and/or a movement of the ultrasound probe 10 and to tilt the transmission directions of the ultrasound waves based on the detected force and/or movement. In other words, the controlling unit 170 is configured to further detect a direction into which the operator (e.g., a laboratory technician) is trying to tilt the transmission directions of the ultrasound waves based on the detected force and/or movement and to tilt the transmission directions of the ultrasound waves based on the detected direction. With this arrangement, the ultrasound diagnosis apparatus 1 according to the first embodiment makes it possible to change the transmission directions of the ultrasound waves without requiring ultrasound jelly or an operation performed on the input device 20.

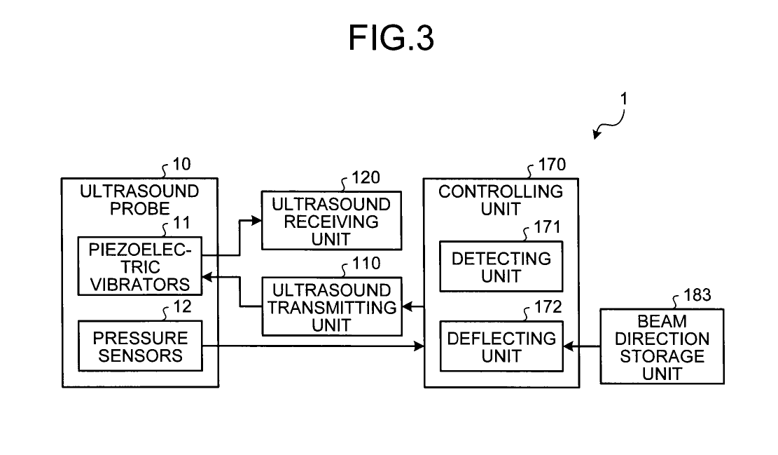

Next, the ultrasound diagnosis apparatus 1 according to the first embodiment configured as described above will be explained in detail, with reference to FIGS. 3 to 6. FIG. 3 is a diagram of an exemplary configuration of the controlling unit 170 and the like according to the first embodiment.

As illustrated in FIG. 3, the ultrasound probe 10 includes piezoelectric vibrators 11 and pressure sensors 12. As explained above, the piezoelectric vibrators 11 generate the ultrasound waves based on the drive signal supplied from the ultrasound transmitting unit 110 and output the received reflected-wave signal to the ultrasound receiving unit 120. Also, as explained above, the transmission delaying unit 112 included in the ultrasound transmitting unit 110 arbitrarily adjusts the transmission directions of the ultrasound waves transmitted from the piezoelectric vibrator surface, by varying the delay periods applied to the rate pulses.

The pressure sensors 12 are provided in the ultrasound probe 10 and serve as a pressure detecting unit configured to detect pressures applied to the ultrasound probe 10. In the first embodiment, one pressure sensor 12 is provided on each of the left and the right ends of the piezoelectric vibrator surface of the ultrasound probe 10. For example, in the ultrasound probe 10 according to the first embodiment, one pressure sensor 12 is provided, for example, in each of point-symmetry positions that are symmetrical with respect to a gravity point position of the piezoelectric vibrator surface. The exemplary embodiments are not limited to this example, and it is acceptable to provide the ultrasound probe 10 with three or more pressure sensors 12.

During an image taking process performed by the ultrasound diagnosis apparatus 1, the pressure sensors 12 perform the process of detecting the pressures at regular time intervals. In other words, each of the pressure sensors 12 detects the force with which the piezoelectric vibrator surface is pressed against the body surface of the patient P, in such a position where the pressure sensor 12 itself is provided on the piezoelectric vibrator surface of the ultrasound probe 10. Further, the pressure sensors 12 sequentially output the pressure values detected in this manner to the ultrasound diagnosis apparatus 1. The pressure values detected by the pressure sensors 12 are sequentially stored into the storage unit 180.

The beam direction storage unit 183 is used in the control exercised by the controlling unit 170, which is explained later. The beam direction storage unit 183 stores therein a pressure difference threshold value and information related to the transmission directions of the ultrasound waves. For example, as the information related to the transmission directions of the ultrasound waves, the beam direction storage unit 183 stores therein a pattern of transmission directions such as “−20°, −10°, 0°, +10°, +20°”.

In the pattern of transmission directions in the present example, a predetermined direction is expressed with “−”, whereas the direction opposite to the predetermined direction is expressed with “+”. For example, the transmission direction of an ultrasound wave transmitted perpendicularly to the piezoelectric vibrator surface is expressed as “0°”. Further, when the piezoelectric vibrator surface is divided into two sections by a straight line passing through the gravity point position of the piezoelectric vibrator surface, “−10°” denotes the transmission direction of the ultrasound wave tilted at “10°” with respect to the transmission direction “0°”, from the gravity point position toward one of the sectioned surfaces. In that situation, “+10°” denotes the transmission direction of the ultrasound wave tilted at “10°” with respect to the transmission direction “0°”, from the gravity point position toward the other sectioned surface. Further, in the pattern of transmission directions “−20°, −10°, 0°, +10°, +20°”, the numerical values shown next to a current value are the transmission direction candidates to which the current value can be changed. For example, if the transmission directions of the ultrasound waves are currently “0°”, the transmission directions after the change are either “−10°” or “+10°”.

How to use various types of information stored in the beam direction storage unit 183 will be explained together with the controlling unit 170. The controlling unit 170 includes a detecting unit 171 and a deflecting unit 172.

Based on a stress or an operation applied to the ultrasound probe 10, the detecting unit 171 detects a direction (hereinafter, this direction may be referred to as a “tilt request direction”) into which the operator who is a laboratory technician is trying to tilt the transmission directions of the ultrasound waves transmitted by the ultrasound probe 10. More specifically, the detecting unit 171 according to the first embodiment detects the force with which the ultrasound probe 10 is pressed against the body surface of the patient P, by using the plurality of pressure sensors 12 provided in the ultrasound probe 10. Further, if the difference between the detected pressures is equal to or larger than the pressure difference threshold value stored in the beam direction storage unit 183, the detecting unit 171 detects, as the tilt request direction, a direction from the position in which the pressure sensor 12 having detected a larger detected pressure is provided, to the position in which the pressure sensor 12 having detected a smaller detected pressure is provided.

The deflecting unit 172 tilts the transmission directions of the ultrasound waves transmitted from the ultrasound probe 10 by a predetermined value into the tilt request direction detected by the detecting unit 171. More specifically, when the tilt request direction has been detected by the detecting unit 171, the deflecting unit 172 tilts the transmission directions of the ultrasound waves by the predetermined value, based on the pattern of transmission directions stored in the beam direction storage unit 183. In this situation, the deflecting unit 172 outputs a delay period to cause the transmission directions of the ultrasound waves to be tilted by the predetermined value, to the ultrasound transmitting unit 110. After that, the transmission delaying unit 112 included in the ultrasound transmitting unit 110 causes the transmission directions of the ultrasound waves to be tilted, by applying the delay period that was input thereto from the deflecting unit 172 to the rate pulses.

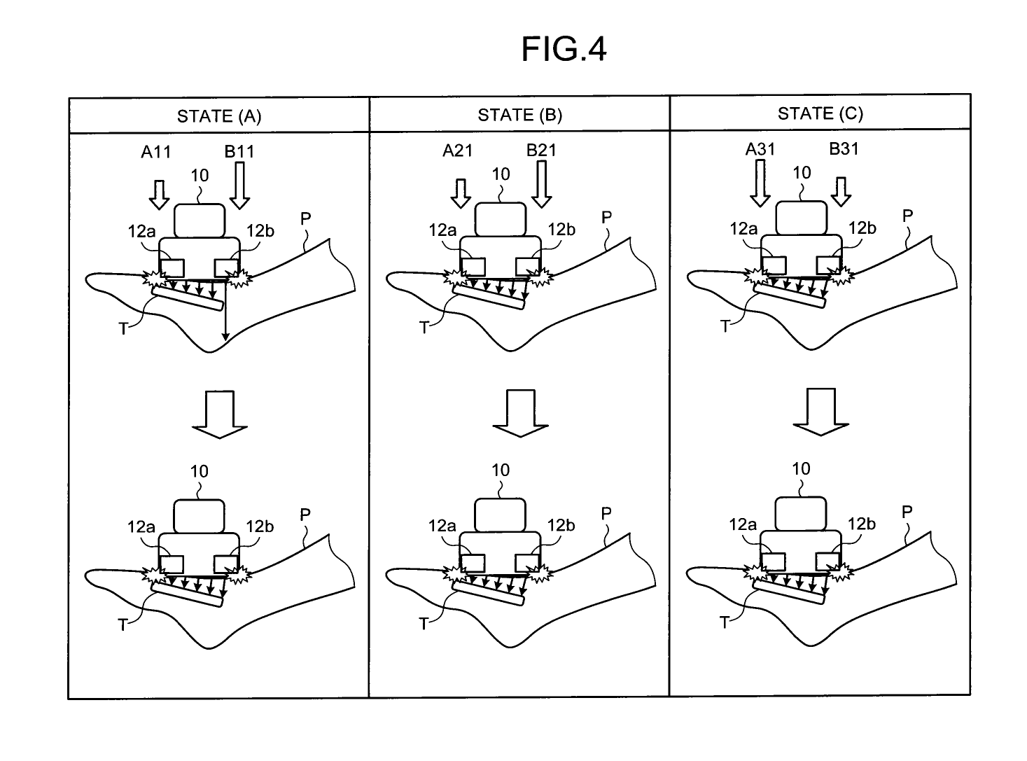

Next, processes performed by the controlling unit 170 according to the first embodiment will be explained, with reference to FIG. 4. FIG. 4 is a drawing of examples of processes performed by the controlling unit 170 according to the first embodiment. In the present example, it is assumed that the beam direction storage unit 183 stores therein “−20°, −10°, 0°, +10°, +20°” as a pattern of transmission directions, like in the example described above. Further, in FIG. 4, it is assumed that the ultrasound waves of which the transmission directions are expressed with a negative (−) angle are transmitted to the left, whereas the ultrasound waves of which the transmission directions are expressed with a positive (+) angle are transmitted to the right.

In the example shown in FIG. 4, the ultrasound probe 10 is provided with a pressure sensor 12a and a pressure sensor 12b that are positioned on the two ends of the piezoelectric vibrator surface. First, let us assume that, as shown in the example in the upper part of State (A) in FIG. 4, the ultrasound probe 10 is transmitting ultrasound waves in the transmission direction “0°”. In other words, the ultrasound probe 10 transmits the ultrasound waves in the directions perpendicular to the piezoelectric vibrator surface. In this situation, let us assume that a pressure A11 detected by the pressure sensor 12a is smaller than a pressure B11 detected by the pressure sensor 12b and that the difference between the pressure A11 and the pressure B11 is equal to or larger than the pressure difference threshold value.

In that situation, it means that the right side (the position in which the pressure sensor 12b is provided) of the ultrasound probe 10 is pressed against the patient P more strongly than the left side (the position in which the pressure sensor 12a is provided) is. In other words, it means that the operator is trying to tilt the ultrasound probe 10 to the right (i.e., a force is applied to the ultrasound probe 10 so as to tilt the ultrasound probe 10 toward the right, or the ultrasound probe 10 is moved so as to tilt toward the right). In other words, it means that the operator is trying to tilt the transmission directions of the ultrasound waves transmitted from the ultrasound probe 10 to the left (i.e., the tilt request direction is the left). Accordingly, the detecting unit 171 detects the direction from the position in which the pressure sensor 12b is provided to the position in which the pressure sensor 12a is provided, as the tilt request direction, which is the direction into which the operator is trying to tilt the transmission directions of the ultrasound waves.

After that, the deflecting unit 172 tilts the transmission directions of the ultrasound waves to the left. More specifically, because the transmission directions of the ultrasound waves transmitted by the ultrasound probe 10 are currently “0°”, while the pattern of transmission directions stored in the beam direction storage unit 183 is “−20°, −10°, 0°, +10°, +20°”, the deflecting unit 172 specifies “−10°” and “+10°”, as transmission direction candidates after the change. Further, because the tilt request direction detected by the detecting unit 171 is the left, the deflecting unit 172 selects “−10°” as the transmission direction after the change, from between the transmission direction candidates “−10°” and “+10°”, and outputs a delay period to cause the transmission directions of the ultrasound waves to be tilted by “−10°”, to the ultrasound transmitting unit 110. As a result, as shown in the example in the lower part of State (A) in FIG. 4, the transmission directions of the ultrasound waves transmitted by the ultrasound probe 10 are the directions tilted to the left by “10°” with respect to a line perpendicular to the piezoelectric vibrator surface.

Subsequently, even after the transmission directions of the ultrasound waves are arranged to be “−10°”, the pressure sensor 12a and the pressure sensor 12b perform the process to detect the pressures. In this situation, let us assume that, as shown in the example in the upper part of State (B) in FIG. 4, a pressure A21 detected by the pressure sensor 12a is smaller than a pressure B21 detected by the pressure sensor 12b and that the difference between the pressure A21 and the pressure B21 is equal to or larger than the pressure difference threshold value.

In that situation, the detecting unit 171 detects the direction from the position in which the pressure sensor 12b is provided to the position in which the pressure sensor 12a is provided as the tilt request direction. Further, because the transmission directions of the ultrasound waves transmitted by the ultrasound probe 10 are currently “−10°”, the deflecting unit 172 outputs a delay period to cause the transmission directions of the ultrasound waves to be tilted by “−20°”, to the ultrasound transmitting unit 110. As a result, as shown in the example in the lower part of State (B) in FIG. 4, the transmission directions of the ultrasound waves transmitted by the ultrasound probe 10 are the directions tilted to the left by “20°” with respect to a line perpendicular to the piezoelectric vibrator surface.

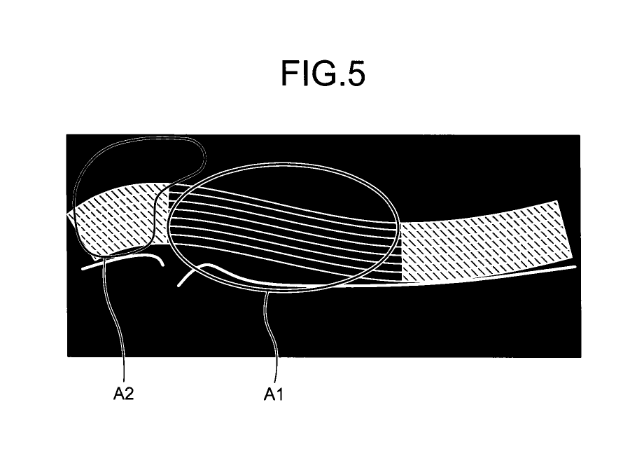

With these arrangements, the ultrasound probe 10 is able to cause the target tissue T to be substantially perpendicularly irradiated by the ultrasound waves. Accordingly, the ultrasound diagnosis apparatus 1 according to the first embodiment is able to generate an ultrasound image rendering the target tissue T clearly. Further, as shown in the example in the lower part of State (B) in FIG. 4, because no gap is formed between the piezoelectric vibrator surface of the ultrasound probe 10 and the body surface, the ultrasound diagnosis apparatus 1 according to the first embodiment is able to generate the ultrasound image that also renders the tissues positioned near the target tissue T. An example of an ultrasound image generated by the ultrasound diagnosis apparatus 1 according to the first embodiment configured as described above is shown in FIG. 5. As shown in FIG. 5, the ultrasound diagnosis apparatus 1 is able to generate an ultrasound image rendering the target tissue T clearly in the area A1 and rendering the tissue positioned near the target tissue T in the area A2.

Even after the transmission directions of the ultrasound waves are arranged to be “−20°”, the pressure sensor 12a and the pressure sensor 12b perform the process to detect the pressures. In this situation, let us assume that, as shown in the example in the upper part of State (C) in FIG. 4, a pressure A31 detected by the pressure sensor 12a is larger than a pressure B31 detected by the pressure sensor 12b and that the difference between the pressure A31 and the pressure B31 is equal to or larger than the pressure difference threshold value. In other words, let us assume that the large/small relationship between the pressure detected by the pressure sensor 12a and the pressure detected by the pressure sensor 12b has become opposite to the large/small relationship in State (B).

In that situation, the detecting unit 171 detects the direction from the position in which the pressure sensor 12a is provided to the position in which the pressure sensor 12b is provided as the tilt request direction. Further, because the transmission directions of the ultrasound waves transmitted by the ultrasound probe 10 are currently “−20°”, the deflecting unit 172 outputs a delay period to cause the transmission directions of the ultrasound waves to be tilted by “−10°”, to the ultrasound transmitting unit 110. As a result, as shown in the example in the lower part of State (C) in FIG. 4, the transmission directions of the ultrasound waves transmitted by the ultrasound probe 10 are the directions tilted to the left by “10°” with respect to a line perpendicular to the piezoelectric vibrator surface. In other words, the transmission directions of the ultrasound waves return to the state shown in the lower part of State (A) in FIG. 4.

It is also acceptable to configure the deflecting unit 172 so as to arrange the transmission directions of the ultrasound waves to be “0°”, instead of “−10°”, in the example shown in State (C) in FIG. 4. In other words, when having changed the transmission directions of the ultrasound waves in one direction and changing the transmission directions again back in the other direction, the deflecting unit 172 may change the transmission directions back to “0°” all at once, instead of gradually changing the transmission directions.

Further, although not shown in FIG. 4, if the operator stops the operation of tilting the ultrasound probe 10 after the situation shown in the lower part of State (B) is achieved, for example, the transmission directions of the ultrasound waves do not change from “−20°”. More specifically, if the operator stops the operation of tilting the ultrasound probe 10, the difference between the pressure detected by the pressure sensor 12a and the pressure detected by the pressure sensor 12b is substantially equal, and the pressure difference between these two pressures is no longer equal to or larger than the pressure difference threshold value. In that situation, because no tilt request direction is detected by the detecting unit 171, the deflecting unit 172 does not perform the process of changing the transmission directions of the ultrasound waves. As a result, the transmission directions of the ultrasound waves transmitted by the ultrasound probe 10 are kept in the deflected state observed immediately prior.

As explained above, the ultrasound diagnosis apparatus 1 according to the first embodiment detects the direction into which the operator is trying to tilt the transmission directions of the ultrasound waves, based on the force with which the ultrasound probe 10 is pressed against the patient P, and changes the transmission directions of the ultrasound waves. As a result, the ultrasound diagnosis apparatus 1 according to the first embodiment is configured so that it is possible to change the transmission directions of the ultrasound waves with the intuitive operation performed by the operator.

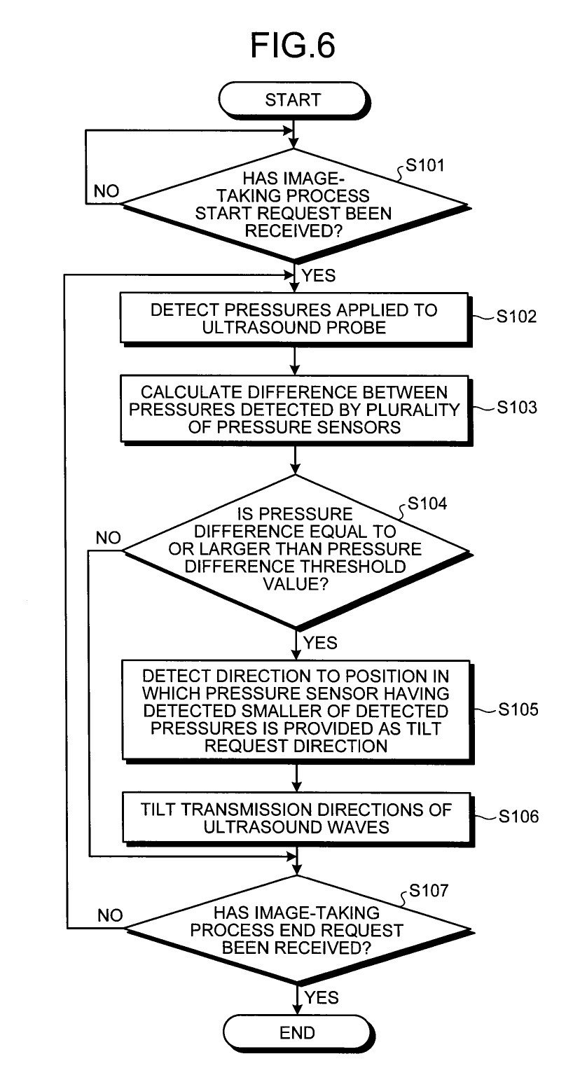

Next, a processing procedure performed by the ultrasound diagnosis apparatus 1 according to the first embodiment will be explained, with reference to FIG. 6. FIG. 6 is a flowchart of the processing procedure performed by the ultrasound diagnosis apparatus 1 according to the first embodiment.

As shown in the example in FIG. 6, the ultrasound diagnosis apparatus 1 judges whether an image-taking process start request has been received from the operator (step S101). In this situation, if no image-taking process start request has been received (step S101: No), the ultrasound diagnosis apparatus 1 stands by until an image-taking process start request is received.

On the contrary, when an image-taking process start request is received (step S101: Yes), the ultrasound diagnosis apparatus 1 starts an image taking process. Although not shown in FIG. 6, the ultrasound diagnosis apparatus 1 performs the process of causing the ultrasound probe 10 to transmit ultrasound waves, the process of generating an ultrasound image based on a reflected-wave signal received by the ultrasound probe 10, and the like, in parallel with the processing procedure at steps S102 through S106 explained below.

In this situation, according to the first embodiment, the plurality of pressure sensors 12 provided in the ultrasound probe 10 detect the pressures applied to the ultrasound probe 10 (step S102). Subsequently, the detecting unit 171 calculates the difference between the pressures detected by the plurality of pressure sensors 12 (step S103). In the first embodiment, because one pressure sensor 12 is provided at each of the left and the right ends of the piezoelectric vibrator surface of the ultrasound probe 10, the detecting unit 171 calculates the difference between the pressures detected by the two pressure sensors 12.

After that, the detecting unit 171 judges whether the calculated pressure difference is equal to or larger than the pressure difference threshold value stored in the beam direction storage unit 183 (step S104). In this situation, if the pressure difference is not equal to or larger than the pressure difference threshold value (step S104: No), the detecting unit 171 ends the process. After that, the ultrasound diagnosis apparatus 1 proceeds to the process at step S107, which is explained later.

On the contrary, if the pressure difference is equal to or larger than the pressure difference threshold value (step S104: Yes), the detecting unit 171 detects the direction from the position in which the pressure sensor having detected the larger of the detected pressures is provided to the position in which the pressure sensor having detected the smaller of the detected pressures is provided, as a tilt request direction, which is the direction into which the operator is trying to tilt the transmission directions of the ultrasound waves (step S105).

Subsequently, the deflecting unit 172 tilts the transmission directions of the ultrasound waves transmitted from the ultrasound probe 10 into the tilt request direction detected by the detecting unit 171 by a predetermined value (step S106). In this situation, the deflecting unit 172 determines the angle at which the transmission directions of the ultrasound waves are tilted, based on the pattern of transmission directions stored in the beam direction storage unit 183 as described above.

After that, the ultrasound diagnosis apparatus 1 judges whether an image-taking process end request has been received from the operator (step S107). In this situation, if no image-taking process end request has been received (step S107: No), the ultrasound diagnosis apparatus 1 returns to the processing procedure at step S102. On the contrary, if an image-taking process end request is received (step S107: Yes), the ultrasound diagnosis apparatus 1 ends the process.

It is acceptable to configure the detecting unit 171 so as to perform the process at step S103 once every predetermined period of time. For example, the detecting unit 171 may calculate the difference between the pressures detected by the two pressure sensors 12 every time a predetermined period of time (e.g., one minute) has elapsed (step S103) and may judge whether the pressure difference is equal to or larger than the pressure difference threshold value (step S104).

As explained above, the ultrasound diagnosis apparatus 1 according to the first embodiment is configured so that it is possible to change the transmission directions of the ultrasound waves with the intuitive operation performed by the operator. As a result, according to the first embodiment, it is possible to generate an ultrasound image rendering the target tissue which the operator desires to view and the tissues positioned near the target tissue, with the intuitive operation performed by the operator.

For example, even in a situation where the ultrasound probe 10 is pressed against such a site where the soft tissue over the bone is thin, because the ultrasound diagnosis apparatus 1 according to the first embodiment is configured to tilt the transmission directions of the ultrasound waves into the direction into which the operator is trying to tilt the ultrasound probe 10, it is possible to cause the target tissue to be substantially perpendicularly irradiated by the ultrasound waves, without forming a gap between the piezoelectric vibrator surface and the body surface. As a result, the ultrasound diagnosis apparatus 1 according to the first embodiment is able to generate the ultrasound image rendering the target tissue and the tissues positioned near the target tissue. Further, because the ultrasound diagnosis apparatus 1 according to the first embodiment is configured so that it is possible to change the transmission directions of the ultrasound waves with the intuitive operation performed by the operator, the ultrasound diagnosis apparatus 1 according to the first embodiment makes it possible to perform the medical examination without using ultrasound jelly and makes it possible to perform the medical examination without operating the input device 20 (e.g., a knob) for the purpose of changing the transmission directions of the ultrasound waves.

In the first embodiment described above, it is also acceptable to configure the ultrasound diagnosis apparatus 1 so as to change the transmission directions of the ultrasound waves, if the operator has performed the operation of trying to tilt the transmission directions of the ultrasound waves for a period of time that is equal to or longer than a predetermined duration. For example, it is acceptable to configure the deflecting unit 172 so as to change the transmission directions of the ultrasound waves, if the detecting unit 171 has continuously detected tilt request directions that are substantially the same as one another for a period of time that is equal to or longer than a predetermined duration. In another example, it is acceptable to configure the deflecting unit 172 so as to change the transmission directions of the ultrasound waves, if the detecting unit 171 has continuously detected tilt request directions that are substantially the same as one another a number of times that is equal to or larger than a predetermined value.

Further, in the first embodiment described above, the example is explained in which the beam direction storage unit 183 stores therein the pattern of transmission directions “−20°, −10°, 0°, +10°, +20°” in which the angle changes by a predetermined amount (10°) at a time. However, the pattern of transmission directions stored in the beam direction storage unit 183 is not limited to this example. For example, the beam direction storage unit 183 may store therein a pattern of transmission directions in which the angle changes irregularly such as “−23°, −20°, −15°, −10°, 0°, +10°, +15°, +20°, +23°”. In this example, the ultrasound diagnosis apparatus 1 changes the transmission directions of the ultrasound waves by larger amounts at the beginning of an image taking process and fine-tunes the transmission directions of the ultrasound waves as the image taking process progresses.

In another example, the beam direction storage unit 183 may simply store therein information such as “10°” as a pattern of transmission directions. In this example, the deflecting unit 172 changes the transmission directions of the ultrasound waves by “10°” at a time, toward the tilt request direction detected by the detecting unit 171.

In yet another example, the beam direction storage unit 183 may store therein information such as “10°/5°/3°”, as a pattern of transmission directions. In that situation, the deflecting unit 172 may change the transmission directions of the ultrasound waves by a large amount when the transmission angle of the ultrasound waves is currently small and may change the transmission directions of the ultrasound waves by a small amount when the transmission angle of the ultrasound waves is currently large. For example, the following arrangement is acceptable: If the transmission directions of the ultrasound waves are currently within a first angle range (e.g., −20° to +20°), the deflecting unit 172 changes the transmission directions of the ultrasound waves by “10°” at a time. If the transmission directions are currently within a second angle range (e.g., “−30° to −20°” or “+20° to +30°”), the deflecting unit 172 changes the transmission directions of the ultrasound waves by “5°” at a time. If the transmission directions are currently within a third angle range (e.g., “−40° to −30°” or “+30° to +40°”), the deflecting unit 172 changes the transmission directions of the ultrasound waves by “3°” at a time.

In yet another example, the following arrangement is also acceptable: If the number of times a tilt request direction is detected by the detecting unit 171 is smaller than a first threshold value, the deflecting unit 172 changes the transmission directions of the ultrasound waves by “10°” at a time. If the number of times of detection is equal to or larger than the first threshold value and is smaller than a second threshold value (which is larger than the first threshold value), the deflecting unit 172 changes the transmission directions of the ultrasound waves by “5°” at a time. If the number of times of detection is equal to or larger than the second threshold value, the deflecting unit 172 changes the transmission directions of the ultrasound waves by “3°” at a time.

Alternatively, the beam direction storage unit 183 may store therein a plurality of pressure difference threshold values. For example, the beam direction storage unit 183 may store therein a first pressure difference threshold value and a second pressure difference threshold value that is larger than the first pressure difference threshold value. In that situation, the detecting unit 171 performs the process of detecting a tilt request direction by using the first pressure difference threshold value, if the transmission angle of the ultrasound waves is currently smaller than a predetermined value, whereas the detecting unit 171 performs the process of detecting a tilt request direction by using the second pressure difference threshold value, if the transmission angle of the ultrasound waves is currently equal to or larger than the predetermined value.

Further, in the first embodiment described above, the example is explained in which the ultrasound probe 10 is configured so that one pressure sensor 12 is provided in each of the left and the right ends of the piezoelectric vibrator surface. However, it is also acceptable to configure the ultrasound probe 10 in such a manner that, for example, one pressure sensor 12 is provided at each of the four corners of the piezoelectric vibrator surface. In that situation, because the pressures are detected by the four pressure sensors 12, the detecting unit 171 is able to three-dimensionally detect the direction into which the operator is trying to tilt the ultrasound probe 10, based on the large/small relationship among the pressures detected by the four pressure sensors 12. In that situation, the detecting unit 171 may virtually tilt the ultrasound probe 10 into the detected direction and detect the direction perpendicular to the piezoelectric vibrator surface of the ultrasound probe 10 that is virtually tilted, as a tilt request direction.

Further, in the first embodiment described above, the example is explained in which the ultrasound probe 10 is provided with the pressure sensors 12. However, another arrangement is also acceptable in which the ultrasound probe 10 is provided with an acceleration sensor, instead of the pressure sensors 12, so that the acceleration sensor detects a movement of the ultrasound probe 10. In this situation, the acceleration sensor operates as a movement detecting unit configured to detect a positional displacement of the ultrasound probe 10. Further, the detecting unit 171 detects the positional displacement of the ultrasound probe 10 through the acceleration sensor and detects a movement direction of the ultrasound probe 10 specified based on the detected positional displacement as a tilt request direction. For example, when the operator performs an operation to slide the ultrasound probe 10 sideways while keeping an end thereof in contact with the body surface of the patient P, the acceleration sensor detects such a positional displacement of the ultrasound probe 10. In that situation, based on the positional displacement of the ultrasound probe 10 detected by the acceleration sensor, the detecting unit 171 detects a movement direction of the ultrasound probe 10 and detects the movement direction as a tilt request direction.

In yet another example, the ultrasound probe 10 may include an acceleration sensor together with the pressure sensors 12. In that situation, the detecting unit 171 detects a tilt request direction based on a detection result obtained by the pressure sensors 12 in the same manner as in the process described above, and also, detects a tilt request direction based on a detection result obtained by the acceleration sensor. In other words, the detecting unit 171 detects the tilt request directions based on the plurality of pieces of information. Thus, the detecting unit 171 is able to detect the direction into which the operator is trying to tilt the transmission directions of the ultrasound waves, with a high level of precision.

### Second Embodiment

In the first embodiment described above, the example is explained in which the tilt request direction is detected based on the difference between the pressures detected by the plurality of pressure sensors provided in the ultrasound probe 10. In a second embodiment, an example will be explained in which, on the premise that sonar gel (a gel pad, a water bag, or the like) is disposed between an ultrasound probe and the body surface, a tilt request direction is detected based on a distance between the ultrasound probe and the body surface.

First, sonar gel will be explained. When taking an image of a site having unevenness (e.g., a thyroid gland) by using an ultrasound diagnosis apparatus, the operator may dispose sonar gel between an ultrasound probe and the body surface. Almost no reflected waves of ultrasound waves occur inside the sonar gel. For this reason, if an ultrasound image is generated by an ultrasound diagnosis apparatus while sonar gel is disposed, the ultrasound image renders the part where the sonar gel and the body surface are in contact with each other, as a high-brightness line that is shaped as a substantially straight line. In the second embodiment, it is assumed that such sonar gel is used during an image taking process performed by an ultrasound diagnosis apparatus.

Next, an ultrasound diagnosis apparatus 2 according to the second embodiment will be explained. Because the configuration of the ultrasound diagnosis apparatus 2 according to the second embodiment is substantially the same as the configuration of the ultrasound diagnosis apparatus 1 shown in FIG. 1, the drawing thereof will be omitted. It should be noted that, however, unlike the ultrasound probe 10 according to the first embodiment, an ultrasound probe according to the second embodiment includes no pressure sensors. Further, a controlling unit according to the second embodiment performs processes different from the processes performed by the controlling unit 170 according to the first embodiment. Furthermore, a beam direction storage unit according to the second embodiment stores therein information different from the information stored in the beam direction storage unit 183 according to the first embodiment.

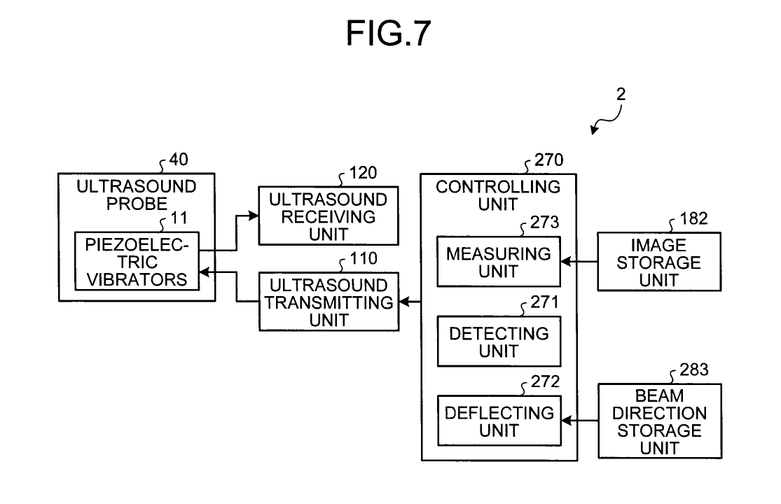

Next, the controlling unit and the like according to the second embodiment will be explained, with reference to FIG. 7. FIG. 7 is a diagram of an exemplary configuration of the controlling unit and the like according to the second embodiment. As shown in FIG. 7, an ultrasound probe 40 according to the second embodiment is a commonly-used ultrasound probe and does not include any sensors such as pressure sensors.

Further, a beam direction storage unit 283 according to the second embodiment is used in the control exercised by a controlling unit 270, which is explained later. The beam direction storage unit 283 stores therein a distance difference threshold value and information related to the transmission directions of the ultrasound waves. For example, the beam direction storage unit 283 stores therein a pattern of transmission directions such as “−20°, −10°, 0°, +10°, +20°”, as the information related to the transmission directions of the ultrasound waves.

Further, the controlling unit 270 according to the second embodiment includes a measuring unit 273, a detecting unit 271, and a deflecting unit 272.

The measuring unit 273 measures the distance from the piezoelectric vibrator surface of the ultrasound probe 40 to the body surface of the patient P, at each of a plurality of locations. More specifically, the measuring unit 273 detects the part where the sonar gel and the body surface are in contact with each other, by analyzing an ultrasound image stored in the image storage unit 182. As described above, because the part where the sonar gel and the body surface are in contact with each other is rendered as a high-brightness line that is shaped as a substantially straight line, the measuring unit 273 is able to detect the contact part as the body surface, by detecting the high-brightness line while using a commonly-used boundary extraction algorithm. Further, the measuring unit 273 measures the distance from the piezoelectric vibrator surface of the ultrasound probe 40 to the body surface, at each of the predetermined plurality of locations within the ultrasound image. For example, the measuring unit 273 measures the distance from the piezoelectric vibrator surface to the body surface at each of the two ends of the piezoelectric vibrator surface.

The detecting unit 271 detects a force applied to the ultrasound probe 40 and/or a movement of the ultrasound probe 40 by using the distances measured by the measuring unit 273 and further detects a tilt request direction, which is a direction into which the operator (who is a laboratory technician) is trying to tilt the transmission directions of the ultrasound waves, based on the detected force and/or movement. More specifically, the detecting unit 271 according to the second embodiment calculates the difference between the distances measured at the plurality of locations by the measuring unit 273 and, if the calculated difference between the distances is equal to or larger than the distance difference threshold value stored in the beam direction storage unit 283, the detecting unit 271 detects the direction from the location having a shorter distance to the location having a longer distance as the tilt request direction.

The deflecting unit 272 tilts the transmission directions of the ultrasound waves transmitted from the ultrasound probe 40 by a predetermined value into the tilt request direction detected by the detecting unit 271. More specifically, like the deflecting unit 172 according to the first embodiment, the deflecting unit 272 tilts the transmission directions of the ultrasound waves by the predetermined value, based on the pattern of transmission directions stored in the beam direction storage unit 283.

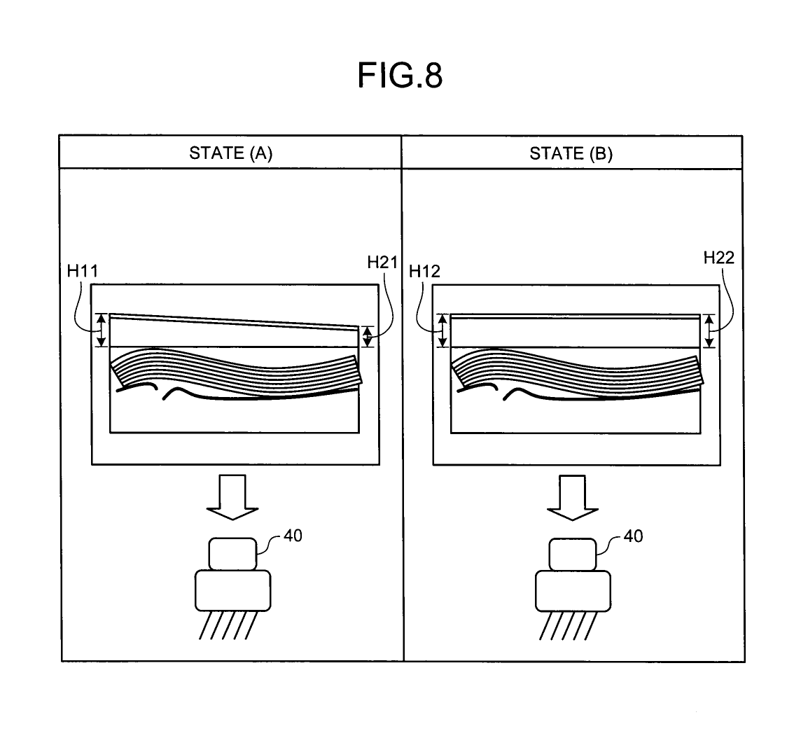

Next, processes performed by the controlling unit 270 according to the second embodiment will be explained, with reference to FIG. 8. FIG. 8 is a drawing of examples of processes performed by the controlling unit 270 according to the second embodiment. In the present example, it is assumed that the beam direction storage unit 283 stores therein “−20°, −10°, 0°, +10°, +20°” as a pattern of transmission directions, like in the example described above. Further, it is assumed that the transmission directions of the ultrasound waves are initially “0°”.

In the example shown in FIG. 8, the measuring unit 273 measures the distance from the piezoelectric vibrator surface to the body surface at each of the two ends of the piezoelectric vibrator surface, by analyzing the ultrasound image. More specifically, in the example shown in the upper part of State (A) in FIG. 8, the measuring unit 273 measures a distance H11 from the left end of the piezoelectric vibrator surface to the body surface and measures a distance H21 from the right end of the piezoelectric vibrator surface to the body surface. In this situation, let us assume that the distance H11 is longer than the distance H21 and that the difference between the distance H11 and the distance H21 is equal to or larger than the distance difference threshold value.

In that situation, it means that the operator is pressing the right side of the ultrasound probe 40 against the patient P more strongly than the left side. In other words, it means that the operator is trying to tilt the ultrasound probe 40 to the right. In other words, it means that the operator is trying to tilt the transmission directions of the ultrasound waves to the left. Accordingly, the detecting unit 271 detects the direction from the right end having the shorter of the distances measured by the measuring unit 273 to the left end having the longer of the measured distances, as the tilt request direction, which is the direction into which the operator is trying to tilt the ultrasound probe 40.

After that, the deflecting unit 272 tilts the transmission directions of the ultrasound waves to the left. More specifically, because the transmission directions of the ultrasound waves transmitted by the ultrasound probe 40 are currently “0°”, while the pattern of transmission directions stored in the beam direction storage unit 283 is “−20°, −10°, 0°, +10°, +20°”, the deflecting unit 272 outputs a delay period to cause the transmission directions of the ultrasound waves to be tilted by “−10°”, to the ultrasound transmitting unit 110. As a result, as shown in the example in the lower part of State (A) in FIG. 8, the transmission directions of the ultrasound waves transmitted by the ultrasound probe 40 are the directions tilted to the left by “10°” with respect to a line perpendicular to the piezoelectric vibrator surface.

Subsequently, even if the operator stops the operation of tilting the ultrasound probe 40 after the transmission directions of the ultrasound waves are arranged to be “−10°”, the transmission directions of the ultrasound waves do not change from “−10°”. More specifically, as shown in the example in the upper part of State (B) in FIG. 8, the measuring unit 273 measures a distance H12 from the left end of the piezoelectric vibrator surface to the body surface and measures a distance H22 from the right end of the piezoelectric vibrator surface to the body surface. In this situation, if the operator is not performing the operation of tilting the ultrasound probe 40, the difference between the distance H12 and the distance H22 is not equal to or larger than the distance difference threshold value. Accordingly, because no tilt request direction is detected by the detecting unit 271, the deflecting unit 272 does not perform the process of tilting the transmission directions of the ultrasound waves. As a result, as shown in the example in the lower part of State (B) in FIG. 8, the transmission directions of the ultrasound waves transmitted by the ultrasound probe 40 remain to be the direction tilted to the left by “10°” with respect to a line perpendicular to the piezoelectric vibrator surface. If the operator wishes to change the transmission directions of the ultrasound waves to “0°” or “+10°”, the operator is able to do so by tilting the ultrasound probe 40 to the left.

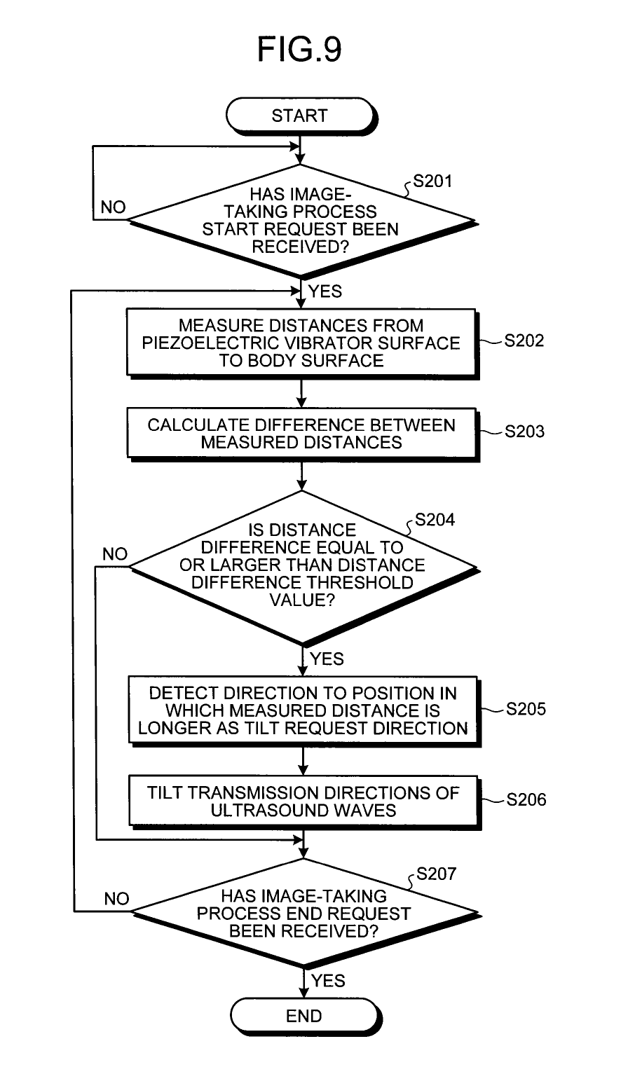

Next, a processing procedure performed by the ultrasound diagnosis apparatus 2 according to the second embodiment will be explained, with reference to FIG. 9. FIG. 9 is a flowchart of the processing procedure performed by the ultrasound diagnosis apparatus 2 according to the second embodiment.

As shown in the example in FIG. 9, the ultrasound diagnosis apparatus 2 judges whether an image-taking process start request has been received from the operator (step S201). In this situation, if no image-taking process start request has been received (step S201: No), the ultrasound diagnosis apparatus 2 stands by until an image-taking process start request is received.

On the contrary, when an image-taking process start request is received (step S201: Yes), the ultrasound diagnosis apparatus 2 starts an image taking process. Although not shown in FIG. 9, the ultrasound diagnosis apparatus 2 performs the process of causing the ultrasound probe 40 to transmit ultrasound waves, the process of generating an ultrasound image based on a reflected-wave signal received by the ultrasound probe 40, and the like, in parallel with the processing procedure at steps S202 through S206 explained below.

In this situation, according to the second embodiment, after at least one ultrasound image is generated by the image generating unit 140, the measuring unit 273 obtains the ultrasound image from the image storage unit 182 and measures the distance from the piezoelectric vibrator surface to the body surface at each of a plurality of locations, by analyzing the obtained ultrasound image (step S202).

Subsequently, the detecting unit 271 calculates the difference between the distances measured by the measuring unit 273 in the plurality of locations (step S203). Further, the detecting unit 271 judges whether the calculated difference between the distances is equal to or larger than the distance difference threshold value stored in the beam direction storage unit 283 (step S204). In this situation, if the distance difference is not equal to or larger than the distance difference threshold value (step S204: No), the detecting unit 271 ends the process. After that, the ultrasound diagnosis apparatus 2 proceeds to the process at step S207, which is explained later.

On the contrary, if the distance difference is equal to or larger than the distance difference threshold value (step S204: Yes), the detecting unit 271 detects the direction from the location having a shorter distance measured by the measuring unit 273 to the location having a longer measured distance, as a tilt request direction (step S205).

Subsequently, the deflecting unit 272 tilts the transmission directions of the ultrasound waves into the tilt request direction detected by the detecting unit 271 by a predetermined value (step S206). In this situation, the deflecting unit 272 determines the angle at which the transmission directions of the ultrasound waves are tilted, based on the pattern of transmission directions stored in the beam direction storage unit 283 as described above.

After that, the ultrasound diagnosis apparatus 2 judges whether an image-taking process end request has been received from the operator (step S207). In this situation, if no image-taking process end request has been received (step S207: No), the ultrasound diagnosis apparatus 2 returns to the processing procedure at step S202. On the contrary, if an image-taking process end request is received (step S207: Yes), the ultrasound diagnosis apparatus 2 ends the process.

It is acceptable to configure the detecting unit 271 so as to perform the process at step S202 once every predetermined period of time. For example, the detecting unit 271 may measure the distance from the piezoelectric vibrator surface to the body surface at each of the plurality of locations every time a predetermined period of time (e.g., one minute) has elapsed, by analyzing the newest ultrasound image stored in the image storage unit 182 (step S202).

As explained above, the ultrasound diagnosis apparatus 2 according to the second embodiment is configured so that it is possible to change the transmission directions of the ultrasound waves with the intuitive operation performed by the operator. As a result, according to the second embodiment, it is possible to generate an ultrasound image rendering the target tissue which the operator desires to view and the tissues positioned near the target tissue, with the intuitive operation performed by the operator.

In the second embodiment described above, it is acceptable, like in the first embodiment, to configure the ultrasound diagnosis apparatus 2 so as to change the transmission directions of the ultrasound waves, if the operator has performed the operation of trying to tilt the transmission directions of the ultrasound waves for a period of time that is equal to or longer than a predetermined duration. For example, it is acceptable to configure the deflecting unit 272 so as to change the transmission directions of the ultrasound waves, if the detecting unit 271 has continuously detected tilt request directions that are the same as one another for a period of time that is equal to or longer than a predetermined duration, or if the detecting unit 271 has continuously detected tilt request directions that are the same as one another a number of times that is equal to or larger than a predetermined value.

Further, like the beam direction storage unit 183, the beam direction storage unit 283 may store therein a pattern of transmission directions in which the angle changes irregularly or may simply store therein a pattern of transmission directions such as “10°” or may store therein a pattern of transmission directions such as “10°/5°/3°”.

Further, in the second embodiment described above, the example is explained in which the measuring unit 273 measures the distance from the left end of the piezoelectric vibrator surface to the body surface and the distance from the right end of the piezoelectric vibrator surface to the body surface. However, it is acceptable to configure the measuring unit 273 to measure the distance from the piezoelectric vibrator surface to the body surface at each of three or more locations. In that situation, the detecting unit 271 may, for example, divide the piezoelectric vibrator surface into two sections with a straight line passing through the gravity point position of the piezoelectric vibrator surface and calculate the difference between an average of the distances from the one of the sectioned piezoelectric vibrator surfaces to the body surface and an average of the distances from the other sectioned piezoelectric vibrator surface to the body surface. Further, in another example, the detecting unit 271 may three-dimensionally detect the direction into which the operator is trying to tilt the ultrasound probe 40 based on a long/short relationship among the distances measured by the measuring unit 273 at three or more locations and may virtually tilt the ultrasound probe 40 into the detected direction and detect the direction perpendicular to the piezoelectric vibrator surface of the ultrasound probe 40 that is virtually tilted, as a tilt request direction.

Furthermore, in the second embodiment described above, it is acceptable to configure the detecting unit 271 so as to calculate a temporal change in the distances measured by the measuring unit 273 for each of the plurality of locations. More specifically, when a distance from the piezoelectric vibrator surface to the body surface has been measured by the measuring unit 273, the detecting unit 271 calculates the difference between the measured distance and a distance from the piezoelectric vibrator surface to the body surface measured by the measuring unit 273 in the immediately preceding measuring process, for each of the plurality of locations. After that, the detecting unit 271 detects the direction into which the operator is trying to tilt the ultrasound probe 40 based on the temporal changes in the measured distances and detects a tilt request direction based on the detection result.

The process described above can be explained with reference to the examples shown in FIG. 8 as follows: When the ultrasound image changes from State (A) to State (B), the detecting unit 271 calculates “H11-H12” as a temporal change in the distance from the left end of the piezoelectric vibrator surface to the body surface and calculates “H21-H22” as a temporal change in the distance from the right end of the piezoelectric vibrator surface to the body surface. In that situation, it means that the operator is trying to tilt the ultrasound probe 40 to the left. In other words, it means that the operator is trying to tilt the transmission directions of the ultrasound waves to the right. Accordingly, the detecting unit 271 detects the direction from the left end to the right end of the piezoelectric vibrator surface as a tilt request direction.

### Other Exemplary Embodiments

The embodiments disclosed herein are not limited to the exemplary embodiments described above. It is possible to implement the embodiments in other various modes.

Magnetic Sensor

In the exemplary embodiments described above, the method is explained as an example by which the movement of the ultrasound probe 10 is detected by detecting the positional displacement of the ultrasound probe 10 while using the acceleration sensor provided in the ultrasound probe 10, so that the tilt request direction is detected based on the detected positional displacement. However, the exemplary embodiments are not limited to this example. For instance, it is acceptable to detect the movement of the ultrasound probe 10 by detecting a positional displacement of the ultrasound probe 10 while using a magnetic sensor provided in the ultrasound probe 10.

When the magnetic sensor is used, the ultrasound diagnosis apparatus 1 includes, for example, a position information obtaining device (not shown). The magnetic sensor provided in the ultrasound probe 10 is configured to detect a three-dimensional magnetic field formed while using a transmitter of the position information obtaining device as the origin, to convert information of the detected magnetic field into a signal, and to output the signal resulting from the conversion to the position information obtaining device. Based on the signal received from the magnetic sensor, the position information obtaining device calculates position coordinates and an orientation direction of the magnetic sensor within the three-dimensional space having the transmitter as the origin and sends the calculated position coordinates and orientation direction to the controlling unit 170. For example, when the operator performs an operation to slide the ultrasound probe 10 sideways while keeping an end thereof in contact with the body surface of the patient P, the detecting unit 171 detects a change in the position coordinates of the magnetic sensor. After that, for example, when a set of position coordinates A have changed to another set of position coordinates B, the detecting unit 171 detects the positional displacement and detects the direction from the set of position coordinates A to the set of position coordinates B as a tilt request direction.

Combinations of Various Types of Methods

In the exemplary embodiments described above, the method using the pressure sensors, the method using the acceleration sensor, the method using the result of the image analysis performed on the ultrasound image, the method using the magnetic sensor, and the like are explained. It is also acceptable to calculate the transmission directions of the ultrasound waves, by selecting any of these methods as appropriate and using the selected methods in combination. For example, it is acceptable to configure the detecting unit so as to obtain a tilt request direction by using three parameters such as the “pressures” detected by the pressure sensors, the “positional displacement” detected by the acceleration sensor, and the “distance” from the ultrasound probe to the body surface obtained by performing an image analysis on an ultrasound image.

Examples of methods for handling a plurality of parameters include a method by which priority levels of the parameters are set and a method by which an average value of results obtained with the parameters is used. When the method by which the priority levels are set is used, for example, the detecting unit sets the priority levels among the parameters and detects a tilt request direction based on parameters having higher priority levels among the parameters yielding values equal to or higher than a threshold value. For example, based on a large/small relationship among the pressures detected by the plurality of pressure sensors and based on the difference among the distances measured at the plurality of locations, the detecting unit is able to three-dimensionally detect the force and the movement applied to the ultrasound probe. Further, in that situation, the detecting unit is able to virtually tilt the ultrasound probe into the detected direction and to detect the direction perpendicular to the piezoelectric vibrator surface of the ultrasound probe that is virtually tilted, as a tilt request direction. In that situation, by using the plurality of parameters, the detecting unit is able to obtain a tilt request direction based on each of the parameters. Accordingly, based on the priority levels that are set in advance, the detecting unit may select one of the tilt request directions obtained from the parameter having a high priority level.

Further, when using the method by which an average value of results obtained with the parameters is used, the detecting unit may, for example, calculate an average value of the tilt request directions obtained by using the parameters and may use the average value as a tilt request direction.

Relationship Between a Tilt Request Direction and the Transmission Directions of the Ultrasound Waves

In the exemplary embodiments described above, the deflecting unit changes the transmission directions of the ultrasound waves according to the pattern of transmission directions. For example, if the tilt request direction detected by the detecting unit is the “left”, the deflecting unit refers to the transmission pattern and determines “−10°” to be the transmission direction after the change, based on the current transmission direction (e.g., “0°”) and the “left”. In this manner, when the detecting unit has detected the rough tilt request direction, the deflecting unit tilts the transmission directions according to the predetermined transmission pattern. In other words, the transmission directions are not necessarily calculated from the tilt request direction. However, the embodiments disclosed herein are not limited to this example.

For example, in the situation where, as explained above, the detecting unit is able to detect the direction perpendicular to the piezoelectric vibrator surface of the ultrasound probe that is virtually tilted as a tilt request direction, the tilt request direction can be obtained as a three-dimensional vector. Accordingly, the deflecting unit may determine the same direction as the tilt request direction to be the transmission directions of the ultrasound waves or may obtain the transmission directions of the ultrasound waves by performing a calculation on the tilt request direction. Further, in the situation where, as explained above, a plurality of parameters are used, the deflecting unit may set priority levels or may use an average value, when calculating the transmission directions while using a plurality of tilt request directions.

Example in which No Tilt Request Direction is Detected

Further, in the exemplary embodiments described above, the method is explained by which, after the detecting unit has detected the tilt request direction, the deflecting unit tilts the transmission directions of the ultrasound waves based on the tilt request direction. However, the exemplary embodiments are not limited to this example. For instance, it is possible to omit the process of detecting the tilt request direction itself by, for example, storing in advance detection patterns of pressure sensors and the directions into which the transmission directions of the ultrasound waves are tilted, in correspondence with one another. In that situation, for example, it is possible to omit the processes at step S105 in FIG. 6 and at step S205 in FIG. 9. For example, the deflecting unit may directly specify the transmission directions of the ultrasound waves by using a detection pattern of the pressure sensors detected by the detecting unit.

When an ultrasound diagnosis apparatus and an ultrasound probe controlling method according to at least one of the embodiments described above is used, it is possible to change the transmission directions of the ultrasound waves with the intuitive operation performed by the operator.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
a detecting unit configured to detect at least one selected from between a force applied to an ultrasound probe and a movement of the ultrasound probe; and
a deflecting unit configured to tilt a transmission direction of an ultrasound wave transmitted from the ultrasound probe, based on at least one selected from between the force and the movement.

2. The ultrasound diagnosis apparatus according to claim 1, wherein the detecting unit detects at least one selected from among a force with which the ultrasound probe is pressed against a surface of a body of a patient, a positional displacement of the ultrasound probe, and a distance from the ultrasound probe to the surface of the body.

3. The ultrasound diagnosis apparatus according to claim 1, wherein the detecting unit detects the distance from the ultrasound probe to the surface of the body by performing an image analysis on an ultrasound image.

4. The ultrasound diagnosis apparatus according to claim 1, wherein
the detecting unit further detects a direction into which an operating person is trying to tilt the transmission direction of the ultrasound wave, based on at least one selected from between the force and the movement, and
the deflecting unit tilts the transmission direction based on the detected direction.

5. The ultrasound diagnosis apparatus according to claim 4, wherein
the ultrasound probe is provided with a plurality of pressure detecting units each of which is configured to detect a pressure applied to the patient from the ultrasound probe, and
based on a difference between the detected pressures detected by the plurality of pressure detecting units, the detecting unit detects a direction from a position in which the pressure detecting unit having detected a larger detected pressure is provided, to a position in which the pressure detecting unit having detected a smaller detected pressure is provided.

6. The ultrasound diagnosis apparatus according to claim 4, wherein
the ultrasound probe includes a movement detecting unit configured to detect a positional displacement of the ultrasound probe, and
the detecting unit detects a movement direction of the ultrasound probe, based on the positional displacement of the ultrasound probe detected by the movement detecting unit.

7. The ultrasound diagnosis apparatus according to claim 4, further comprising: a measuring unit configured to measure a distance from a vibrator surface of the ultrasound probe to the surface of the body of the patient at each of a plurality of locations, wherein
based on the measured distances measured by the measuring unit at the plurality of locations, the detecting unit detects a direction from a location having a shorter measured distance, to a location having a longer measured distance.

8. The ultrasound diagnosis apparatus according to claim 7, wherein
the measuring unit performs the process of measuring the distance between the surface of the body and the vibrator surface at each of the plurality of locations once every predetermined period of time, and
the detecting unit detects a direction from a location having a smaller temporal change in the measured distance measured by the measuring unit, to a location having a larger temporal change in the measured distance.

9. The ultrasound diagnosis apparatus according to claim 4, wherein, when the detecting unit has continuously detected directions that are substantially same as one another for a predetermined period of time or when the detecting unit has continuously detected directions that are substantially same as one another a predetermined number of times, the deflecting unit tilts the transmission direction of the ultrasound wave transmitted from the ultrasound probe based on the detected directions.

10. An ultrasound diagnosis apparatus comprising:
an ultrasound probe; and
a deflecting unit configured to tilt a transmission direction of an ultrasound wave transmitted from the ultrasound probe, based on at least one selected from among a force with which the ultrasound probe is pressed against a surface of a body of a patient, a positional displacement of the ultrasound probe, and a distance from the ultrasound probe to the surface of the body.

11. A method for controlling an ultrasound probe implemented by an ultrasound diagnosis apparatus, the method comprising:
a detecting step of detecting at least one selected from among a force with which the ultrasound probe is pressed against a surface of a body of a patient, a positional displacement of the ultrasound probe, and a distance from the ultrasound probe to the surface of the body; and
a deflecting step of tilting a transmission direction of an ultrasound wave transmitted from the ultrasound probe, based on at least one selected from among the force with which the ultrasound probe is pressed against the surface of the body of the patient, the positional displacement of the ultrasound probe, and the distance from the ultrasound probe to the surface of the body.
